(19) United States
(12) Patent Application Publication
(10) Pub. No.: US 2020/0000641 A1
(43) Pub. Date: Jan. 2, 2020

(54) THERMALLY-CONDUCTIVE, METAL-BASED BANDAGES WITH HYDROGEL SUBSTRATE

(71) Applicant: HARRISBURG (B.V.I) LIMITED, Tortola (VG)

(72) Inventor: Stephen J. CARROLL, Liverpool (GB)

(21) Appl. No.: 16/508,904

(22) Filed: Jul. 11, 2019

Related U.S. Application Data
(63) Continuation of application No. 15/262,107, filed on Sep. 12, 2016, now abandoned.
Application No. 15/262,107 is a continuation of application No. 14/440,130, filed on May 1, 2015, now abandoned.
(60) Provisional application No. 61/723,075, filed on Nov. 6, 2012.

(51) Int. Cl. A61F 13/00; A61F 13/02

(52) U.S. Cl. CPC A61F 13/00063; A61F 13/022; A61F 13/0213; A61F 13/00051; A61F 2013/00289; A61F 13/00059; A61F 2013/00157; A61F 2013/00187; A61F 13/0226

(57) ABSTRACT

The invention is a class of medical bandages that are effective for use in the treatment of various types of tissue burns, such as burns due to heat, chemicals, or sun exposure. The inventive bandages are comprised of a thin metal substrate in combination with a heat-sink. The inventive bandages incorporate a metal substrate (such as aluminum) having a burn-facing side for direct contact with the burn to draw heat away from the burn by conduction, and a heat-sink facing side opposite the burn-facing side for contact with a hydrogel to draw heat away from the metal layer by conduction. The thin aluminum layer and associated hydrogel heat-sink ensures flexibility and effective heat-transfer characteristics to rapidly cool a burn wound.

### BACKGROUND OF THE INVENTION

Burn injuries are caused by fire, chemicals, electricity, and friction and can vary in severity. First degree burns are the least severe, causing redness, and healing relatively quickly. On the other end of the spectrum, fourth degree burns are the most severe, burning down to the level of the muscle and bone. Second and third degree burns fall between these extremes.

Medical professionals often try to strike a balance when deciding how to treat burns. On one hand, if a burn is superficial and relatively dry, then it may be desirable to keep the wound moist with water or some sort of ointment or cream. However, a problem with applying many ointments and/or creams is that such applications often do not help draw heat away from a wound. On the other hand, if a burn is more serious, such as a second-degree burn that is oozing fluid, then there is an enhanced fear of infection. In such cases, some medical professionals feel that such wounds should be kept relatively dry, while still others may advocate for the application of various ointment dressings with antibiotic properties to fight infection. Hence, it would be desirable to come up with a treatment strategy that is able to provide the best of all worlds.

On Aug. 30, 1948, Time Magazine reported that steam from an exploding locomotive had scalded Fireman Frank Mihlan of the Erie Railroad. When Mihlan was carried into Cleveland's Charity Hospital on Jul. 15, 1948, 70% of his body was burned, and doctors thought that Mihlan had little chance of survival. However, attending surgeons decided to try wrapping the Mihlan's burns in thin strips of aluminum foil, a technique developed by Toronto's Dr. Alfred W. Farmer. It was the first time that aluminum foil for burns had been used in the U.S.; the first time it had ever been used for burns of the whole body. Relief from pain was “miraculous”, and within 20 minutes of application, Mihlan was resting comfortably. As an added precaution, Mihlan was given intravenous fluids and penicillin. The aluminum foil, which looked like the inside wrapping of a cigarette package, apparently acted as a seal for the body fluids that seep from burned surfaces. It also apparently helped kill bacteria, speeding the healing process. Twelve days after being bandaged in the aluminum foil wrappings, Mihlan was out of bed. Eventually, Mihlan left the hospital unscarred, albeit temporarily reddened.

Bandages and wraps may incorporate a thin layer of thermally conductive metal (such as aluminum) at the base of a substrate adapted to be in direct contact with a burn wound, while the top side of the aluminum substrate has a heat-dissipation-enhancing topography to help cool burns faster by enhancing thermal convection properties. Such products are described in Aluminaid's U.S. Pat. No. 8,530,720 to Freer, et al. Heat from a burn will be drawn from the burn to the metal substrate through conduction. Aluminum does not effectively store conducted heat but is an excellent conductor of heat. Aluminum conducts heat away from the source and readily gives the heat up to its surrounding atmospheric environment through convection.

Certain heat-dissipation-enhancing-topographies of the thermally conductive layer may have technically complicated designs or may be difficult to manufacture—cheaply or efficiently. In situations where one wishes to reduce the complexity of the thermally conductive metal's topography, it may be desirable to incorporate into a thermally conductive bandage a more efficient method of heat transfer away from a burn via conduction, rather than convection. When one side of a thermally conductive bandage is applied to a burn, an additional layer of material may be present on the opposite side of the conductive bandage substrate to act as a heat sink. This additional layer will act as a heat sink into which heat can be removed from the burn area and stored, or further dissipated into the atmospheric environment through convection. Hydrogel may act as a convenient heat sink in such applications.

It would be advantageous to develop a bandage having aluminum or other thermally conductive material as a conductive substrate in combination with a hydrogel substrate so that heat may be rapidly drawn away from the burn by the aluminum, and then further drawn away from the aluminum into the hydrogel heat sink. Such a bandage would effectively cool a subject's burn and further alleviate pain associated with subject's burn.

### SUMMARY OF THE INVENTION

The invention is a class of medical products designed to alleviate discomfort and relieve pain caused by burns. The inventive bandage includes a layer of thermally conductive metal (particularly aluminum) bonded with a heat sink (particularly a hydrogel). A bandage incorporating a hydrogel as a primary cooling agent, bonded with an aluminum substrate, would improve thermal transfer from a subject's burn to a thermal heat sink.

The inventive bandage is configured to orient the aluminum substrate directly onto the subject's burn to draw heat away from the burn through conduction. Bonded to the opposite side of the aluminum substrate is a hydrogel which draws heat away from the aluminum.

There are two principal actions occurring in the inventive bandage when applied to a burn. First is thermal transfer into the aluminum substrate from the wound via conduction. Second is thermal transfer out of the aluminum substrate and into a hydrogel layer which acts as a thermal reservoir. Maximizing the thermal reservoir increases the rate of cooling. The thin aluminum layer and associated hydrogel heat-sink ensures flexibility and effective heat-transfer characteristics to rapidly cool a burn wound.

Hydrogels are networks of hydrophilic polymer chains in which water is the dispersion medium. Hydrogels are mostly water, and some have over 99% water. Hydrogels generally exhibit flexibility similar to that of human tissue due to their substantial water content. Water has a high specific heat capacity, and a hydrogel having a large water content will similarly have a high specific heat capacity. High specific heat capacity, coupled with physical flexibility and biocompatible nature, make hydrogels a preferred choice for a heat sink in the inventive bandage.

The inventive bandage is secured over a subject's burn with a top layer of adhesive material adapted for use on the subject. A removable backing layer adhered to the very bottom of the bandage protects the adhesive material and the burn-contacting portions of the bandage until the backing layer is removed from the bandage for use. It is preferred that the bandage components are thin and flexible to enhance patient comfort.

Methods of using the inventive bandage include facilitating and expediting heat-dissipation from a burn to assist in the healing of a burn. It is a goal of the invention to achieve thermal equilibrium between the burn and the bandage within about fifteen (15) to about 300, more preferably within about 15 to about 120 seconds. It is a goal of the invention to alleviate, reduce, and eliminate symptoms of burn within about fifteen (15) to about 300 seconds of bandage application.

These and other features, aspects and advantages of the present invention will become better understood with reference to the following drawings, description, and claims.

### BRIEF DESCRIPTION OF THE DRAWINGS

FIG. 1 shows an expanded assembly of a bandage including a bottom backing layer, metal thermal radiator layer, hydrogel absorber layer, and top adhesive layer.

FIG. 2 shows a cut-away schematic of an assembled bandage indicating the position and sizes of the layers.

FIG. 3 depicts a cross-sectional view of the layers within the assembled bandage shown in FIG. 2.

### DETAILED DESCRIPTION OF THE INVENTION

There are three ways in which thermal energy transfer can be described:
Conduction; Convection; and Radiation. Conduction requires physical contact (similar to the flow of electricity in wire). Convection emanates from the movement of molecules (e.g., the way in which heated and cooled water or other fluid moves up and down). Radiation does not necessarily involve direct contact (e.g., the way the sun emits light rays).

At any given temperature, a given mass of aluminum holds much less energy than an equivalent mass of human flesh. For instance, in convection or conduction, if one touches aluminum foil from an oven during the cooking process, a subject's hand and the foil share the thermal energy. The hand (of much greater mass) requires much more energy to raise its temperature (if at all, depending upon the physical connection between the foil and the food). When the subject touches aluminum foil, the foil transfers heat to the flesh; however, due to the aluminum's low specific-heat capacity, the foil quickly loses energy, barely raising the temperature of the skin in contact. Because aluminum foil does not effectively store conducted heat it therefore facilitates the “cooling” of a burn.

Aluminum is non-toxic and used widely in the medical industry. While aluminum does not effectively store conducted heat, aluminum is nonetheless an excellent conductor of heat. Aluminum conducts heat away from the source and readily gives the heat up to its surroundings. This has a cooling effect to the source of the heat. Aluminum can be an effective conductor of a subject's body heat, alleviating pain which emanates from added warmth on a subject's burn. Aluminum metal is generally unreactive and non-toxic, and aluminum will resist adhering to a burn wound—these properties permit aluminum to conduct heat away from the burn without negatively interfering with natural wound healing processes.

Convection generally has significantly lower thermal transfer effects than conduction. Conduction can transfer hundreds or even thousands of times more thermal energy than convection. For planar wall conduction—when the non-controllable variables are removed—the thermal transfer is directly proportional to the thermal conductivity multiplied by the contact area, divided by the wall thickness. For convection—when the non-controllable variables are removed—the thermal transfer is directly proportional to the contact area. Minimizing material thickness and optimizing thermal conductivity are expected to transfer thermal energy at a rate thousands of times faster through conduction than via convection.

The bandages of the invention utilize a layer of thermally conductive metal to draw heat from the burn via conduction, and utilize a heat sink to draw heat from the metal (and again away from the burn) via conduction. The thermally conductive metal substrate and heat sink are physically coupled to ensure efficient conduction of heat from the burn to the heat sink. The inventive bandages are designed to swiftly and efficiently alleviate discomfort and pain caused by burns including those resulting from sun exposure, fire, chemicals, electricity, or friction.

The inventive bandages contain a thin substrate of a thermally conductive metal. Various metals or alloys may be used in the inventive bandages and preferred metals or alloys are those with efficient heat-transfer qualities. Metals or metal alloys may also be chosen based on additional qualities such as biocompatibility, chemical reactivity, or machinability. A particularly preferred metal aluminum because of its thermal conductivity.

Preferred thermally conductive metals include aluminum, silver, gold, copper, zinc, magnesium, tungsten, titanium, and platinum. Other preferred metals include iron, nickel, zinc, tin, and palladium. In one preferred embodiment the metal is aluminum. Preferably the metal contains 98.00% minimum aluminum. In one embodiment aluminum ASTM B479 1 145 is used due to its ease of procurement in sizeable manufacturing quantity.

Alloys substantially based on these metals and other biocompatible metal alloys may also be used. Such alloys include aluminum alloys, chromium/molybdenum/iron alloys, or aluminum/magnesium alloys. One preferred aluminum alloy contains at least about 90% aluminum. One preferred aluminum alloy contains at least 92% aluminum and about 5% magnesium. Other metals can be used in specific quantities to fulfill a specific requirement of wound care.

One layer of metal or more than one layer of metal suitably bonded may be used in the metal substrate. In one embodiment a layer of aluminum and a layer of copper are bonded to form the thermally conductive layer. In one embodiment a layer of aluminum-clad copper is used.

The metal or metal alloy in the invention is preferably sized as a thin sheet or foil. As the metal thickness is increased, conductive performance is reduced. Additionally, as the metal thickness is increased, the bandage will increase in rigidity due to the increased force required for deformation. However, as the metal thickness is reduced, machinability and foil integrity may be reduced. The metal or metal alloy in the inventive bandage may be annealed to enhance the ductility and flexibility of the metal layer.

The metal or metal alloy preferably has a thickness in the range from about 0.00025 inches to about 0.006 inches. In one embodiment the metal or metal alloy layer is about 0.0005 inches to about 0.005 inches thick. The metal may be about 0.0005 inches, about 0.0010 inches, about 0.0015 inches, about 0.0020 inches, about 0.0025 inches, about 0.0030 inches, about 0.0035 inches, about 0.0040 inches, about 0.0045 inches or about 0.0050 inches thick. In one embodiment, the metal is about 0.0005 inches thick. In one embodiment, the metal is about 0.0020 inches thick. In a preferred embodiment, the metal is about 0.0010 inches thick. In one embodiment, the metal substrate layer is about 0.0010 inches thick.

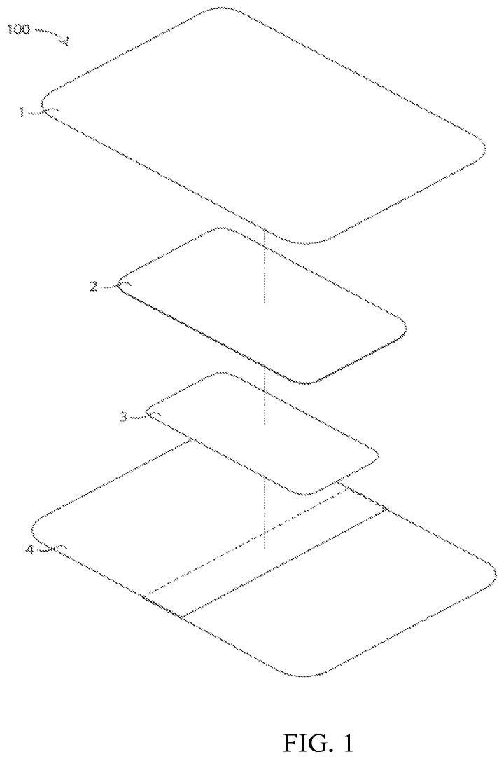

In one embodiment the metal or metal alloy layer is substantially flat. In another embodiment the metal or metal alloy layer is textured to increase the surface area of metal in contact with the heat-sink and thus increase the efficiency of heat transfer. In one embodiment the metal layer is an aluminum sheet or foil. In one embodiment the metal layer is a sheet that has on one side a substantially smooth surface; in one embodiment the metal layer is a sheet that has on one side a dull, matte or brushed surface. In one embodiment the metal layer is an aluminum sheet that has on one side a textured surface having a plurality of discrete protrusions as depicted in FIGS. 9A-9B, 1 OA-101, 1 1B, 12A-12B of Aluminaid's U.S. Pat. No. 8,530,720 to Freer, et al.

In an embodiment where the metal layer is a substantially smooth sheet or foil, the metal substrate has a thickness in the range from about 0.00025 inches to about 0.006 inches. In an embodiment where the metal layer has a plurality of discrete protrusions, the metal substrate has a thickness of about 0.00025 inches to about 0.040 inches as measured from the bottom side of the metal substrate to the average peak height of the plurality of protrusions on the top side of the metal substrate.

The inventive bandages contain a heat sink coupled to the thermally conductive metal layer. A preferred heat-sink is a hydrogel substrate that is flexible, biocompatible, and acts as a thermal reservoir. Hydrogel may act as a convenient heat sink in the inventive bandages in part because of the high specific heat of water (4.186 Joules/(grams×degree Kelvin)). Preferred hydrogels have a high water content and a high specific heat capacity. One preferred hydrogel contains glycerol and water. Suitable hydrogels for use with the inventive bandages may be obtained from commercial sources. In one embodiment, the hydrogel has a specific heat capacity of greater than about 2 Joules/(grams×degree Kelvin). In one embodiment, the hydrogel has a specific heat capacity of greater than about 3 Joules/(grams×degree Kelvin). In one embodiment, the hydrogel has a specific heat capacity of greater than about 4 Joules/(grams×degree Kelvin). The conductive metal layer and hydrogel layer may be bonded together by the adhesive properties of the hydrogel and may also be bonded together by the addition of an adhesive.

The hydrogel substrate is preferably sized as a thin sheet. Maximizing the hydrogel thermal reservoir increases the rate of cooling, but as the hydrogel thickness is increased the bandage will increase in rigidity. However, as the hydrogel thickness is reduced, thermal capacity may be reduced. The hydrogel substrate is preferably in the range from about 0.005 inches to about 0.100 inches thick. In one embodiment the hydrogel layer is about 0.005 inches to about 0.050 inches thick. The hydrogel may be about 0.005 inches, about 0.010 inches, about 0.015 inches, about 0.020 inches, about 0.025 inches, about 0.030 inches, about 0.035 inches, about 0.040 inches, about 0.045 inches, about 0.050, about 0.055 inches, about 0.060 inches, about 0.065 inches, about 0.070 inches, about 0.075 inches, about 0.080 inches, about 0.085 inches, about 0.090 inches, about 0.095 inches, or about 0.100 inches in thickness. In one embodiment, the hydrogel is about 0.030 inches thick. In one embodiment, the hydrogel layer is about 0.015 inches thick. In one embodiment, the hydrogel layer is about 0.010 inches thick.

In a preferred embodiment they hydrogel substrate is sized larger than the metal substrate; in a preferred embodiment the perimeter of the hydrogel layer completely surrounds the perimeter of the metal layer. Ideally, the metal heat spreader is designed to transfer heat from a burn wound that has considerably smaller surface area when compared to that of the bandage. The metal layer spreads the elevated burn's added heat across the entire surface of the hydrogel layer providing greater surface area for conduction contact and, in turn, reduced time until thermal equilibrium is reached between the burn and the hydrogel. This benefit reduces the burn temperature swiftly without significantly affecting the equilibrium temperature. Further, when a bandage that is sized larger than the size of a burn wound is applied to the burn, the time required to reach thermal equilibrium is reduced as a result of lateral heat propagation.

In a preferred embodiment, the metal layer is sized to completely cover the burn to avoid direct contact of the hydrogel to the burn area. Such a bandage would eliminate negative adhesive properties of applying a hydrogel directly to a burn which is common in commercial hydrogel products. Such a bandage would further benefit from the thermal conduction aspects of aluminum for heat-spreading purposes. Such a bandage would effectively cool a subject's burn and further alleviate pain associated with subject's burn.

The hydrogel substrate may be about 1.1 times to about 3.0 times the size of the metal layer substrate. The hydrogel substrate may be about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 times the size of the metal layer substrate.

In one embodiment, the ratio of the area of the hydrogel substrate to the area of the metal substrate is about 3.36:2.00—where the hydrogel substrate is about 1.68 times larger than the metal substrate. In one embodiment, the ratio of the area of the hydrogel substrate to the area of the metal substrate is about 8.16:6.00—where the hydrogel substrate is about 1.36 times larger than the metal substrate. In one embodiment, the ratio of the area of the hydrogel substrate to the area of the metal substrate is about 12.76:10.00—where the hydrogel substrate is about 1.28 times larger than the metal substrate. In one embodiment, the ratio of the area of the hydrogel substrate to the area of the metal substrate is about 1.11:0.45—where the hydrogel substrate is about 2.46 times larger than the metal substrate. In one embodiment, the ratio of the area of the hydrogel substrate to the area of the metal substrate is about 1.28:0.56—where the hydrogel substrate is about 2.27 times larger than the metal substrate. In one example the inventive bandage includes a substantially rectangular metal layer having dimensions of about 2.00 inches by about 1.00 inches, and substantially rectangular hydrogel layer having dimensions of about 2.40 inches by about 1.4 inches.

The hydrogel layer of the inventive bandage is coupled to a top adhesive layer which extends beyond the boundaries of the hydrogel layer. The top adhesive layer is a thin film and may be made of a polymeric material. The top adhesive layer has adhesive material disposed on the bottom surface to facilitate coupling to a subject's skin, and a top surface that is adhesive-free. Polymer medical tape may be used as the top adhesive layer. A selection of materials commonly used in medical bandages may be used as the top adhesive layer. A perforated polymer such as 1527-ENP ethylene vinyl acetate (EVA) is preferred in one embodiment. In one embodiment commercially available medical tape is used as the top adhesive layer.

A removable bandage-backing layer, or release liner, is disposed across the entire bottom surface of the bandage and is coupled to the bandage via the adhesive present in the top adhesive layer. The removable backing layer is detachably coupled to the adhesive top layer so as to be readily peeled away from the bandage. In one embodiment the backing layer extends slightly beyond the boundaries of the top adhesive layer; in one embodiment the backing layer has substantially the same surface area as the top adhesive layer and the backing layer is positioned to be flush with the top adhesive layer. The backing layer is made of a material that can adhere to the top adhesive layer during manufacturing, packaging, and storage, yet can be readily removed from the bandage when desired so as to free the bandage for application to a subject's burn.

In one embodiment the backing layer comprises two or more sheets. In one embodiment, the backing layer consists of two partially overlapping sheets. In this embodiment the each sheet may be partially in contact with the bandage, and partially in contact with the other sheet.

In one embodiment, the top adhesive layer, hydrogel substrate and metal substrate of the inventive bandage are concentric to one another. In another embodiment, the hydrogel substrate and metal substrate are concentric to each other and are positioned so as to be off-center from the top adhesive layer within the inventive bandage. In one embodiment the entire top surface of the hydrogel substrate is in contact with the bottom surface of the top adhesive layer; in one embodiment the entire top surface of the metal substrate is in contact with the bottom surface of the hydrogel substrate. In one embodiment the backing layer is in contact with the bottom surface of the inventive bandage such that the backing layer contacts a portion of the bottom surface of the top adhesive layer, a portion of the bottom surface of the hydrogel substrate, and the entire bottom surface of the metal substrate.

The inventive bandage can be further enhanced by the inclusion of a thermochromic indicator member, wherein the thermochromic indicator member is in thermal communication with a burn wound via the top adhesive layer. A thermochromic compound—similar to what is typically found in mood rings—provides visual feedback regarding the heat removed from the subject's burn. The thermochromic indicator member is comprised of material calibrated to indicate when a burn on which said bandage is applied is still too warm for safe removal of said bandage, based on a predetermined threshold, and indicate when a burn has cooled to at least a predetermined threshold such that said bandage can be safely removed and/or changed-out for a new medical dressing.

In one embodiment the thermochromic indicator member provides color-based indications as to the thermal status of the burn to which said bandage is applied. In another embodiment the thermochromic indicator member provides icon-based indications as to the thermal status of the burn to which the bandage is applied. In some applications, the thermochromic indicator member is comprised of material selected from the group consisting of thermochromic liquid crystals, leuco dyes, and thermochromic inks.

In one embodiment the metal substrate has an extended member that extends beyond the border of the coupled hydrogel layer to be under, and in direct contact with the thermochromic compound present in the top adhesive layer such that the metal extension provides thermal communication between a burn and the thermochromic compound. In one embodiment the thermochromic indicators have compounds calibrated to indicate when a burn is sufficiently cooled (for example by providing a color indicator such as green and/or an icon indicator such as a happy face) or still too warm (for example by providing a color indicator such as red and/or an icon indicator such as sad face). In one embodiment the inventive bandage has a thermochromic compound that does not present a visible color at room temperature; upon application of the bandage to a burn the thermochromic compound turns red (indicating the subject should keep the bandage in place); after time passes and the burned tissue cools the thermochromic compound turns green (indicating the subject may remove the bandage).

In one embodiment the thermochromic indicator changes color on the end closest to the metal substrate more quickly than the end farthest from the metal substrate due a temperature gradient across the indicator. Stratification of the color change of the thermochromic indicator provides indication regarding the rate and amount of cooling.

The inventive bandage may take a variety of forms. In a preferred embodiment the inventive bandage is substantially rectangular; in another embodiment the inventive bandage is substantially square. In one embodiment the inventive bandage is substantially elliptical; in another embodiment the inventive bandage is substantially ovular; in yet another embodiment the inventive bandage is substantially circular. In one embodiment the inventive bandage is substantially triangular; in one embodiment the inventive bandage is substantially trapezoidal. In one embodiment the inventive bandage is substantially heart-shaped. In yet another embodiment the inventive bandage is substantially octagonal. The inventive bandage may be bow-tie shaped or butterfly shaped. The inventive bandages may have corners that are squared or rounded.

The inventive bandage may be shaped to conform to different body contours and body parts such as a glove- or mitt-shape for comfortable use on a burned hand, or an H-shaped bandage to wrap comfortably around a burned finger. The inventive bandage form-factor may be adapted to facilitate application to a part of the body selected from the group consisting of finger, thumb, toe, wrist, elbow, knee, ankle, foot, hand, palm and face.

FIG. 1 depicts an expanded view of the components of one embodiment of the inventive bandage 100. Top adhesive layer 1 has is substantially rectangular with rounded corners. Top adhesive layer 1 has adhesive material disposed on the bottom surface and a top surface that is adhesive-free. The top surface of top adhesive layer 1 may include text and graphics printed on the surface, or may further include a thermochromic indicator. Underneath the top adhesive layer 1 is the hydrogel layer 2. Hydrogel layer 2 is sized to be smaller than top adhesive layer 1 so that top adhesive layer 1 completely covers hydrogel layer 2. Underneath hydrogel layer 2 is the thermally conductive metal layer 3. Metal layer 3 is sized to be smaller than hydrogel layer 2 so that hydrogel layer 2 completely covers metal layer 3. Bandage-backing layer 4 is disposed across the entire bottom surface of the inventive bandage 100 and is sized to be slightly larger than, and substantially the same shape as, top adhesive layer 1. Backing layer 4 comprises two partially overlapping sheets—the two sheets are sized and oriented to ensure complete coverage of the inventive bandage 100 whose largest surface is top layer 1.

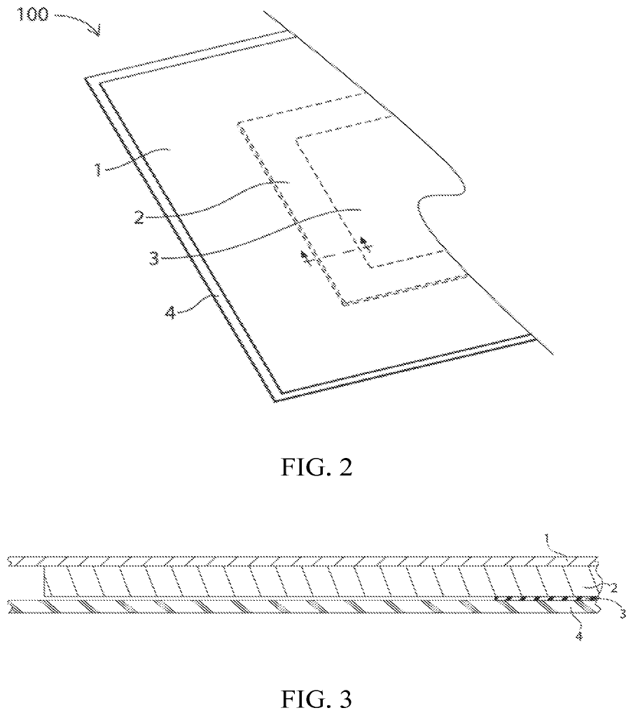

FIG. 2 depicts a schematic of one embodiment of the inventive bandage 100 showing the relative positions of top adhesive layer 1, hydrogel layer 2, and metal layer 3, along with backing layer 4. In the inventive bandage 100, the adhesive surface of top adhesive layer 1 is coupled to the top side of hydrogel layer 2; the bottom side of hydrogel layer 2 is coupled to the top side of the metal layer 3; and the inventive bandage 100 further includes a removable backing layer 4 coupled to the bottom surface of the bandage.

FIG. 3 depicts a cross-sectional view of the inventive bandage 100 of FIG. 2. As shown in FIG. 3, the entire top side of hydrogel layer 2 is in contact with the bottom side of adhesive layer 1. Further, the entire top side of metal layer 3 is in contact with the bottom side of hydrogel layer 2. Backing layer 4 is depicted as contacting the bottom side of metal layer 3, but backing layer 4 will also contact a portion of the bottom side of hydrogel layer 2 as well as a portion of the bottom side of adhesive layer 1.

Additional components may also be included with the bandage such as antibacterial agents to suppress bacterial growth and assist with wound healing or anesthetics and analgesics to reduce pain. Antibacterial agents may include metal ions (such as silver ions) or metal salts (such as silver nitrate, lactate or citrate, or aluminum diacetate), metal nanoparticles (such as silver nanoparticles), sulfates and silvers, antibacterial peptides, quaternary ammonium compounds, triclosan, iodine, PVP-iodine, phenol compounds, chlorhexidine gluconate, polyhexamide, silver sulfadiazine, octenidine, as well as antibiotics such as sulfate, beta-lactams, fluoroquinolones, aminoglycosides, glycopeptides, oxazolidinones, bacteriocin, or tetracycline. Anesthetics and analgesics may include lidocaine, benzocaine, procaine, aloe, menthol, paracetamol, non-steroidal anti-inflammatory drugs and opioid drugs. In one embodiment heparan sulfate is included in the burn dressing a promoter of wound healing. In one embodiment heparan derived glycosaminoglycans including dermatan sulfate, keratan sulfate, chondroitin-4 and chondroitin-6-sulfate, and hyaluronic acid can be added to accelerate wound healing.

Depending on the type and severity of burn, in addition to the inventive bandage a thermally conductive adhesive, paste, gel, or grease may be applied to the area of a subject's skin to enhance the heat transfer from a burn wound to the thermally-conductive metal layer. In some of these variations, the thermally conductive compound is derived from metal or silicone (usually with a zinc-oxide or aluminum-oxide inclusion to improve conductivity), and may fill gaps where air would normally be present. The thermally conductive compound provides a superior conductor (as compared to air) almost equal to that of the conductor itself. The performance of thermally conductive compound is measured in W/m-K. Standard silicon/zinc-oxide thermal compound has thermal conductivities in the range of 0.7-0.9 W/m-K. In such variations, the thermally conductive medium used can also be an aluminum-infused medicinal/therapeutic cream, ointment, or other compound.

While the present inventions have been illustrated and described in many embodiments of varying scope, it will at once be apparent to those skilled in the art that variations may be made within the spirit and scope of the inventions. Accordingly, it is intended that the scope of the inventions set forth in the appended claims not be limited by any specific wording in the foregoing description, except as expressly provided.

### EXAMPLES

### Example 1

The following example is meant to be illustrative and prophetic only. In this example, an inventive bandage is comprised of a top adhesive layer, a hydrogel layer, an aluminum layer, and a backing layer. The top adhesive layer is substantially rectangular and has dimensions of about 3.4 inches by about 2.4 inches with a thickness of about 0.0044 inches. The top adhesive layer is made of commercially available medical tape.

Coupled to the top adhesive layer is a hydrogel layer that is substantially rectangular having dimensions of about 2.3 inches by about 1.3 inches with a thickness of about 0.015 inches. The hydrogel layer is made of commercially available hydrogel.

Coupled to the hydrogel layer is an aluminum layer. The aluminum layer is substantially rectangular and has dimensions of about 2.0 inches by about 1.0 inches with a thickness of about 0.001 inches. The aluminum is a sheet conforming to ASTM B479 1 145.

Finally a backing layer is coupled to the bandage. The backing layer is substantially rectangular having dimensions of about 3.4 inches by about 2.4 inches with a thickness of about 0.0061 inches. The backing layer is comprised of two equally sized sheets each about 1.9 inches by about 2.4 inches—the sheets overlap each other by about 0.5 inches to facilitate removal from the bandage.

### Example 2

The following example is meant to be illustrative and prophetic only. In this example, an inventive bandage of Example 1 is applied to a burn. The aluminum layer draws heat away from the burn via conduction and transfers the thermal energy via conduction to the hydrogel layer. Within about 15 to about 120 seconds, thermal equilibrium is reached between the burn and the hydrogel substrate, and the discomfort and pain caused by the burn are reduced.

1. A bandage comprising:
(a) a top layer of polymeric material, the top layer having a first surface and a second surface, and where the second surface of the top layer has adhesive disposed thereon;
(b) a middle layer of hydrogel substrate, the hydrogel substrate having a first surface and a second surface, where the first surface of the hydrogel substrate is coupled to the second surface of the top layer, and where the hydrogel substrate is positioned within the perimeter of the top layer, wherein said hydrogel has a specific heat capacity of greater than 2 Joules/(grams×degree Kelvin), and
(c) a bottom layer of metal substrate, the metal substrate having a first surface and a second surface, where the first surface of the metal substrate is coupled to the second surface of the hydrogel substrate and where the metal substrate is positioned within the perimeter of the hydrogel substrate,
wherein the entire first surface of the metal substrate is in contact with the second surface of the hydrogel substrate so that the hydrogel substrate completely covers the metal substrate.

2. The bandage of claim 1, wherein said metal comprises an aluminum.

3. The bandage of claim 2, wherein said aluminum is 0.001 inches thick.

4. The bandage of claim 3, further comprising a backing layer removably coupled to a bottom surface of the bandage.

5. The bandage of claim 1, further comprising a thermochromic indicator member disposed within the top layer.

6. The bandage of claim 1, wherein the hydrogel substrate is 1.1 times to 3.0 times the size of the metal substrate.

7. The bandage of claim 1, wherein the first surface of the metal substrate includes a plurality of protrusions and wherein the second surface of the metal substrate is smooth.

8. The bandage of claim 1, wherein the metal is a smooth sheet.

9. A triple layered bandage, said triple layered bandage comprising:
a top layer comprising a polymeric material with a top surface and a bottom surface, wherein said bottom surface has adhesive disposed thereon;
a bottom layer comprising a metal having a top surface and a bottom surface; and
a middle layer disposed between said top layer and said bottom layer comprising hydrogel having a top surface and a bottom surface,
wherein the entire top surface of the bottom layer is in contact with the bottom surface of the middle layer so that the hydrogel completely covers the metal, wherein said hydrogel has a specific heat capacity of greater than 2 Joules/(grams×degree Kelvin).

10. The triple layered bandage of claim 9, wherein said metal comprises aluminum.

11. The triple layered bandage of claim 9, wherein said aluminum is 0.001 inches thick.

12. The triple layered bandage of claim 9, further comprising a thermochromic indicator member disposed within the top layer.

13. The triple layered bandage of claim 9, wherein the hydrogel middle layer is 1.1 times to 3.0 times the size of the metal bottom layer.

14. The triple layered bandage of claim 9, wherein the top surface of the metal bottom layer includes a plurality of protrusions and wherein the bottom surface of the metal bottom layer is smooth.

15. The triple layered bandage of claim 9, wherein the metal is a smooth sheet.
